(19) United States
(12) Patent Application Publication
(10) Pub. No.: US 2003/0077564 A1
(43) Pub. Date: Apr. 24, 2003

(54) Nutrient medium for maintaining neural cells in injured nervous system

(75) Inventor: Gregory J. Brewer, Springfield, IL (US)

Correspondence Address: FITCH EVEN TABIN AND FLANNERY, 120 SOUTH LA SALLE STREET, SUITE 1600, CHICAGO, IL, 60603-3406, US

(73) Assignee: Board of Trustees of Southern Illinois University

(21) Appl. No.: 10/261,462

(22) Filed: Sep. 30, 2002

Related U.S. Application Data
(60) Provisional application No. 60/326,658, filed on Oct. 2, 2001.

(51) Int. Cl. A01N 1/00; A61K 35/30

(52) U.S. Cl. 435/1.1; 424/93.7; 435/374

(57) ABSTRACT

A method to improve neural cell viability in brain or spinal cord tissue after brain or spinal cord injury or surgery is provided. This method comprises applying a sterile liquid medium to the brain or spinal cord tissue, wherein the sterile aqueous liquid medium comprises 0 to about 3000 μM CaCl₂, about 0.1 to about 1.2 μM Fe(NO₃)₃, about 2500 to about 10000 μM KCl, 0 to about 4000 μM MgCl₂, about 30000 to about 150000 μM NaCl, about 100 to about 30000 μM NaHCO₃, about 250 to about 4000 μM NaH₂PO₄, about 0.01 to about 0.4 μM sodium selenite, about 0.2 to about 2 μM ZnSO₄, about 2500 to about 50000 μM D-glucose, about 1 to about 50 μM L-carnitine, about 3 to about 80 μM ethanolamine, about 15 to about 400 μM D(+)-galactose, about 40 to about 800 μM putrescine, about 20 to about 500 μM sodium pyruvate, and growth-promoting essential fatty acids, hormones, amino acids, vitamins and anti-oxidants in amounts effective for neuron growth, and wherein the medium is essentially free of ferrous sulfate, glutamate, and aspartate.

### RELATED APPLICATION

This application is based on and claims the benefit under 35 U.S.C. §119(e) of U.S. Provisional Patent Application No. 60/326,658 filed on Oct. 2, 2001, by Brewer, entitled “Nutrient Medium for Maintaining Neural Cells in Injured Nervous System,” which is hereby incorporated by reference in its entirety.

### FIELD OF THE INVENTION

The current invention relates to an improved aqueous medium for maintaining viability of exposed, injured, or isolated neural cells. The current invention also relates to improved methods for maintaining viability of exposed, injured, or isolated neural cells. The current invention also relates to methods for using the improved culture medium in neurosurgery for human patients.

### BACKGROUND

A major problem attendant to studies of injured central nervous system tissue is the maintenance of cell viability. The inability to maintain central nervous system tissue viability in culture for prolonged periods of time and under various environmental conditions has impeded the development of effective therapeutic regimens for treating central nervous system disorders.

A nutrient balanced salt solution (medium) for maintaining central nervous system tissue viability in a high-carbon dioxide atmosphere (5 percent CO₂) has recently been developed. Neurobasal™ (Gibco/lnvitrogen, Inc., Rockville, Md.) is a bicarbonate buffered medium optimized for the growth of embryonic rat hippocampal neurons at a pH of 7.3 in 5 percent CO₂. This medium is a derivative of Dulbecco's Modified Eagle's Medium (DMEM) and was formulated to optimize embryonic rat hippocampal cell survival. When compared to DMEM, Neurobasal™ has less NaCl and less NaHCO₃, resulting in a lower osmolarity, and lesser amounts of cysteine and glutamine, resulting in diminished glial growth. In addition, Neurobasal™ contains alanine, asparagine, proline, and vitamin B12, all of which are absent from DMEM.

Although neurons can be maintained in a 5 percent CO₂ atmosphere in this high bicarbonate medium, when supplemented with B27 (a hormone and anti-oxidant supplement available from Invitrogen, Inc.), neurons undergo rapid death when transferred to ambient CO₂ conditions (about 0.2 percent). Death is associated with a rapid rise in medium pH to,a value of about 8.1.

The preparation and study of neural tissue and cells frequently requires the use of ambient CO₂ levels outside of an incubator. Existing methods for controlling the pH of cells outside of incubators include the use of weak buffers (e.g., as found in Dulbecco's modified Eagle's medium or L 15 medium) and the use of continuous gassing with 5-10 percent CO₂ to maintain physiological pH. A simple test, however, shows that ambient CO₂ causes the pH of DMEM to quickly rise to a value of about 8.1 outside the incubator. The common practice of buffering with HEPES slows, but does not prevent, this substantial alkalinization. The practice of continuously gassing tissues to maintain high CO₂ levels and physiological pH is cumbersome and expensive.

U.S. Pat. No. 6,180,404 (Jan. 30, 2001), which is owned by the same assignee as the present application and which is hereby incorporated herein by reference, provides a culture medium for maintaining neural cells in ambient CO₂ conditions. The culture medium contains less than about 2000 μM bicarbonate, a buffer having a pKₐ of about 6.9 to about 7.7, from 0 to about 3000 μM CaCl₂, from about 0.05 to about 0.8 μM Fe(NO₃)₃, from about 2500 to about 10000 μM KCl, from 0 to about 4000 μM MgCl₂, from about 74000 to about 103000 μM NaCl, from about 400 to about 2000 μM NaHCO₃, from about 250 to about 4000 μM NaH₂PO₄, from about 0.2 to about 2 μM ZnSO₄, from about 2500 to about 50000 μM D-glucose, and from about 20 to about 500 μM sodium pyruvate, and wherein the medium is free of ferrous sulfate, glutamate, and aspartate. A version of this medium is available commercially under the tradename Hibernate™. Preferably, the medium is supplemented with B27, a growth-promoting supplement that contains effective amounts of hormones, essential fatty acids, and anti-oxidants for neural cells.

There remains a need in the art for an improved medium that can maintain physiological pH and can provide improved neural cell viability in injured brain and spinal cord tissue where a damaged blood supply may reduce CO₂ and other nutrients, hormones, and/or growth factors that promote regeneration and/or prevent or significantly reduce degeneration. The present invention provides such an improved medium.

Brain tumors are the second leading cause of cancer death in children under the age of 15 and in young adults up to the age of 34. Brain tumors are the second fastest growing cause of cancer death in adults over the age of 65. Unlike many other cancers, behavioral modifications do not appear to significantly reduce the risk of such brain cancers. Although about 40 to about 50 percent of brain cancers are benign, benign brain cancers may still result in significant impairment and death. Approximately 100,000 persons in the United States per year are expected to be diagnosed with a primary or metastatic brain tumor. Where the location of the tumor allows, surgical techniques are often used to attempt to remove the brain tumor. The surgical site (i.e., subarachnoid spaces, brain parenchyma, and resection cavity) is generally rinsed with normal saline and sometimes packed with materials soaked in normal saline. Thus, there remains a need for an improved medium and methods which can be used in neurosurgery to improve neural cell viability and/or improve neural cell regeneration and/or improve neural cell differentiation. Such an improved medium and method could be used to rinse or instill the surgical site and/or used to impregnate or saturate a filling material (e.g., Gel-Foam™ sponge) intended to remain in the cavity after removal of the tumor or other nerve tissue. The present invention provides such an improved medium and method.

### SUMMARY OF THE INVENTION

In one aspect, the present invention provides a method to improve neural cell viability in brain or spinal cord tissue after brain or spinal cord injury or surgery in a human, said method comprising applying a sterile aqueous liquid medium to the brain or spinal cord tissue, wherein the medium comprises 0 to about 3000 μM CaCl₂, about 0.1 to about 1.2 μM Fe(NO₃)₃, about 2500 to about 10000 μM KCl, 0 to about 4000 μM MgCl₂, about 30000 to about 150000 μM NaCl, about 100 to about 30000 μM NaHCO₃, about 250 to about 4000 μM NaH₂PO₄, about 0.01 to about 0.4 μM sodium selenite, about 0.2 to about 2 μM ZnSO₄, about 2500 to about 50000 μM D-glucose, about 1 to about 50 μM L-carnitine, about 3 to about 80 μM ethanolamine, about 15 to about 400 μM D(+)-galactose, about 40 to about 800 μM putrescine, about 20 to about 500 μM sodium pyruvate, and growth-promoting essential fatty acids, hormones, amino acids, vitamins and anti-oxidants in amounts effective for neuron growth, and wherein the medium is essentially free of ferrous sulfate, glutamate, and aspartate. The sterile liquid medium may also contain an effective amount of dehydroepiandrosterone-4-sulfate (DHEAS) to maintain hormone levels, generally, an effective amount of DHEAS is about 2 to about 200 μM and, more preferably, about 5 to about 100 μM. The sterile liquid medium may also contain an effective amount of basic fibroblast growth factor (basic FGF or FGF2) to assist in supporting survival and regeneration of neurons; generally an effective amount of FGF2 is about 1 to about 50 ng/ml and, more preferably, about 2 to about 20 ng/ml. An especially preferred FGF2 for use in the present invention is basic human recombinant fibroblast growth factor from Invitrogen, Inc. (Rockville, Md.). Even more preferably, the sterile liquid medium contains effective amounts of both DHEAS and FGF2. Such a preferred composition will generally contain about 5 to about 50 μM DHEAS and about 1 to about 50 ng/ml FGF2 and, more preferably, about 10 to about 30 μM DHEAS and about 2 to about 20 ng/ml FGF2.

In another aspect, the present invention provides a method for delivering stem cells or nervous system cells or tissue having increased viability into a brain, spinal cord, or nervous system of a human, said method comprising (1) treating the stem cells or nervous system cells or tissue with an aqueous sterile liquid medium prior to or during the delivery of the stem cells or nervous system cells or tissue to the brain, spinal cord, or nervous system of the human and (2) delivering the treated stem cells or nervous system cells or tissue to the brain, spinal cord, or nervous system of the human, wherein the aqueous sterile liquid medium comprises 0 to about 3000 μM CaCl₂, about 0.01 to about 1.2 μM Fe(NO₃)₃, about 2500 to about 10000 μM KCl, 0 to about 4000 μM MgCl₂, about 30000 to about 150000 μM NaCl, about 100 to about 30000 μM NaHCO₃, about 250 to about 4000 μM NaH₂PO₄, about 0.01 to about 0.4 μM sodium selenite, about 0.2 to about 2 μM ZnSO₄, about 2500 to about 50000 μM D-glucose, about 1 to about 50 μM L-carnitine, about 3 to about 80 μM ethanolamine, about 15 to about 400 μM D(+)-galactose, about 40 to about 800 μM putrescine, about 20 to about 500 μM sodium pyruvate, and growth-promoting essential fatty acids, hormones, amino acids, vitamins and anti-oxidants in amounts effective for neuron growth and wherein the aqueous sterile liquid medium has an osmolarity of from about 200 to about 270 mOsm, contains about 5000 to about 25000 μM of a hydrogen ion buffer having a pKₐ of from about 6.9 to about 7.7, and is essentially free of ferrous sulfate, glutamate, and aspartate. The sterile liquid medium may also contain an effective amount of dehydroepiandrosterone-4-sulfate (DHEAS) to maintain hormone levels; generally, an effective amount of DHEAS is about 2 to about 200 μM and, more preferably, about 5 to about 100 μM. The sterile liquid medium may also contain an effective amount of basic fibroblast growth factor (basic FGF or FGF2) to assist in supporting survival and regeneration of neurons; generally an effective amount of FGF2 is about 1 to about 50 ng/ml and, more preferably, about 2 to about 20 ng/ml. An especially preferred FGF2 for use in the present invention is basic human recombinant fibroblast growth factor from Invitrogen, Inc. (Rockville, Md.). Even more preferably, the sterile liquid medium contains effective amounts of both DHEAS and FGF2. Such a preferred composition will generally contain about 5 to about 50 μM DHEAS and about 1 to about 50 ng/ml FGF2 and, more preferably, about 10 to about 30 μM DHEAS and about 2 to about 20 ng/ml FGF2.

In still another aspect, the present invention provides an aqueous composition effective for improving neural cell viability in brain or spinal cord tissue in a human after brain or spinal cord injury or surgery or for improving neural cell viability of nervous system cells or tissue intended to be delivered into a brain, spinal cord, or nervous system of a human, said aqueous composition comprising 0 to about 3000 μM CaCl₂; about 0.1 to about 1.2 μM Fe(NO₃)3; about 2500 to about 10,000 μM KCl; 0 to about 4000 μM MgCl₂; about 30,000 to about 150,000 μM NaCl; about 100 to about 30,000 μM NaHCO₃; about 250 to about 4000 μM NaH₂PO₄; about 0.01 to about 0.4 μM sodium selenite; about 0.2 to about 2 μM ZnSO₄; about 2500 to about 50,000 μM D-glucose; about 1 to about 50 μM L-carnitine; about 3 to about 80 μM ethanolamine; about 15 to about 400 μM D(+)-galactose; about 5 to about 200 μM human albumin; about 40 to about 800 μM putrescine; about 20 to about 500 μM sodium pyruvate; about 0.01 to about 0.32 μM transferrin; 0 to about 120 μM L-alanine; 0 to about 2400 μM L-arginine; 0 to about 30 μM L-asparagine; 0 to about 60 μM L-cysteine; 0 to about 3000 μM L-glutamine; 0 to about 2400 μM glycine; 0 to about 1200 μM L-histidine; 0 to about 5000 μM L-isoleucine; 0 to about 5000 μM L-leucine; 0 to about 5000 μM L-lysine; 0 to about 1200 μM L-methionine; 0 to about 2400 μM L-phenylalanine; 0 to about 500 μM L-proline; 0 to about 2400 μM L-serine; 0 to about 5000 μM L-threonine; 0 to about 500 μM L-tryptophan; 0 to about 2400 μM L-tyrosine; 0 to about 5000 μM L-valine; about 0.5 to about 16 μM glutathione (reduced); about 0.1 to about 10 μM α-tocoperol; about 0.1 to about 10 μM α-tocoperol acetate; about 0.001 to about 0.1 μM catalase; about 0.01 to about 0.5 μM superoxide dismutase; about 0.001 to about 0.1 μM cortisol; 0 to about 200 μM DHEAS; about 0.001 to about 0.1 μM progesterone; about 0.02 to about 1 μM retinyl acetate; about 0.1 to about 5 μM insulin; 0 to about 0.6 μM 3,3′,5-triiodo-L-thyronine (T3); about 0.05 to about 20 μM linoleic acid; about 0.1 to about 10 μM linolenic acid; 0 to about 2.5 μM biotin; 0 to about 100 μM D-Ca pantothenate; 0 to about 200 μM choline chloride; 0 to about 100 μM folic acid; 0 to about 240 μM i-inositol; 0 to about 200 μM niacinamide; 0 to about 120 μM pyridoxal; 0 to about 6 μM riboflavin; 0 to about 100 μM thiamine; and 0 to about 1.2 μM cobalamin; and wherein the aqueous composition has an osmolarity of from about 200 to about 270 mOsm, contains about 5000 to about 25000 μM of a hydrogen ion buffer having a pKₐ of from about 6.9 to about 7.7, and is essentially free of ferrous sulfate, glutamate, and aspartate.

### BRIEF DESCRIPTION OF THE DRAWINGS

FIG. 1 illustrates the synergism of DHEAS and FGF2 in the sterile liquid medium of this invention for survival of human neurons. Neurons were cultured for 6 days in the presence (solid circles) or the absence (open circles) of 10 μM DHEAS with 0 to about 10 ng/ml of basic human recombinant fibroblast growth factor from Invitrogen, Inc. (Rockville, Md.). Values are means and S.E. from 6 fields of 0.3 mm² from a 67 year old primary brain lymphoma case. Multifactor ANOVA F(1, 10)=15.2 for lowest three FGF2 concentrations. Note that the zero concentration is artificially located on the logarithmic x-axis.

FIG. 2, based on Example 2, illustrates that the sterile liquid medium of this invention in gelfoam in a rat fimbria-fornix lesion preserves neuron density of axotomized neurons in the medial septum for at least 1 month. Cell densities in the unlesioned side were 61 and 62 cells/mm² for the saline (control) and inventive sterile liquid medium groups, respectively. A second set of controls (sham—no injury) were also carried out. Means+S.E. from 6 rats per group are shown. Probabilities are t-tests vs. saline.

FIG. 3, based on Example 3, illustrates that the sterile liquid medium of this invention in gelfoam increases 1 month survival of rat cortical neurons surrounding the aspiration lesion. Panel A: Treatment with the sterile liquid medium of this invention (solid circles) is significantly better as compared to animals treated with saline (triangles) and is essentially equivalent to sham brains (unlesioned, open circles). Panel B: Relative neuron density as a percentage of density on the unlesioned side. The basic sterile liquid medium of this invention (solid circles) was compared to a bicarbonate-buffered sterile liquid medium of this invention(squares) and to saline (open circles); generally the basic sterile liquid medium was superior to bicarbonate-buffered sterile liquid medium. Panel C: The sterile liquid medium of this invention+FGF2 (squares) is superior to both DMEM+FGF2 (diamonds) and to saline (triangles). Each point is the mean and S.E. for measures from 6 animals as a function of distance from the edge of the lesion. Probabilities were determined by two-factor ANOVA comparing sterile liquid medium of this invention against saline or against DMEM.

FIG. 4, also based on Example 3, illustrates that the sterile liquid medium of this invention (circles) in gelfoam placed into a cortical aspiration lesion eliminates gliosis compared to saline (squares) and sham (triangles), based on GFAP immunostaining. Each point is the mean and S.E. of pixel intensities in an area 20×400 μm in 20 μm increments from the edge of the lesion at a depth of 1200 μm from the pia from 3 rats for each treatment.

FIG. 5A, based on Example 4, shows meningioma cells grown in Neurobasal A medium with 10% fetal bovine serum (GIBCO) and 0.5 mM glutamine after 7 days in culture. These cells spread onto the substrate and proliferated, reaching a mean cell area of 3015+453 μm² (mean+/−S.E., n=12 cells).

FIG. 5B, based on Example 4, shows that meningioma cells grown in inventive medium after culture for 7 days did not spread or proliferate (mean area=936 μm²)(t-test vs. serum-grown cells, p=0.0001). Almost no live cells remained in the inventive medium.

FIG. 5C, based on Example 4, shows glioblastoma cells grown in Neurobasal A medium with 10% fetal bovine serum (GIBCO) and 0.5 mM glutamine after 7 days in culture. These cells spread onto the substrate and proliferated.

FIG. 5D, based on Example 4, shows that glioblastoma cells grown in inventive medium after culture for 7 days did not spread or proliferate.

FIG. 6, based on Example 4, Table 3, is a graph which illustrates meningioma cell growth in Neurobasal A medium with 10% fetal bovine serum and 0.5 mM glutamine (open circles) versus inventive medium (closed circles). Cell growth for a meningioma case was followed over 10 days. After 10 days in culture, the cells grown in Neurobasal A medium with 10% fetal bovine serum (GIBCO) and 0.5 mM glutamine produced confluent growth. These cells were collected by trypsinization and replated either in Neurobasal A medium with 10% fetal bovine serum (GIBCO) and 0.5 mM glutamine or in inventive medium. Growth continued in Neurobasal A medium with 10% fetal bovine serum (GIBCO) and 0.5 mM glutamine serum, but growth was inhibited, and cells died in the inventive medium.

FIG. 7, based on Example 4, Table 4, shows a comparison of cell growth for various types of tumors in either Neurobasal A with fetal bovine serum (cross hatched bars) or inventive medium (solid bars). Fold increase of cells at either six or seven days, calculated by dividing the number of cells at day 6 or 7 by the cell count at the start of the culture, is shown. For these five consecutive tumor cases, the inventive medium results in growth stasis or inhibition and cell death, while Neurobasal A with fetal bovine serum caused cell proliferation in all primary tumors and cell stasis in the metastasis tumor.

### DESCRIPTION OF THE PREFERRED EMBODIMENTS

The present invention provides a method to improve neural cell viability in brain or spinal cord tissue after brain or spinal cord injury or surgery. Said method comprises applying a sterile liquid aqueous medium to the brain or spinal cord tissue, wherein the medium comprises 0 to about 3000 μM CaCl₂, about 0.1 to about 1.2 μM Fe(NO₃)₃, about 2500 to about 10000 μM KCl, 0 to about 4000 μM MgCl₂, about 30000 to about 150000 μM NaCl, about 100 to about 30000 μM NaHCO₃, about 250 to about 4000 μM NaH₂PO₄, about 0.01 to about 0.4 μM sodium selenite, about 0.2 to about 2 μM ZnSO₄, about 2500 to about 50000 μM D-glucose, about 1 to about 50 μM L-carnitine, about 3 to about 80 μM ethanolamine, about 15 to about 400 μM D(+)-galactose, about 40 to about 800 μM putrescine, about 20 to about 500 μM sodium pyruvate, and essential fatty acids, hormones, and anti-oxidants in amounts effective for neuron growth. The medium used in the invention provides a minimal essential aqueous-based medium for maintaining neural cell or tissue viability in an environment containing ambient levels of CO₂ and generally contains less than about 2000 μM bicarbonate, has an osmolarity of from about 200 to about 270 mOsm, contains a buffer having a pKₐ of from about 6.9 to about 7.7, is essentially free of ferrous sulfate, glutamate, and aspartate. For purposes of this invention, “essentially free of ferrous sulfate, gluatmate, and aspartate” means that the composition contains less than about 0.4 μM ferrous sulfate, less than about 1 μM gluatmate, and less than about 1 μM aspartate; preferably, the compositions contain no added ferrous sulfate, gluatmate, or aspartate; more preferably, the levels of these constituents approach, or are, zero.

Generally, the osmolarity is preferably from about 200 to about 240 mOsm. Preferably, the sterile liquid medium comprises, in final concentration, 500 to about 2500 μM CaCl₂, about 0.05 to about 0.6 μM Fe(NO₃)₃, about 3000 to about 8000 μM KCl, about 300 to about 2000 μM MgCl₂, about 40000 to about 103000 μM NaCl, about 200 to about 1800 μM NaHCO₃, about 400 to about 2000 μM NaH₂PO₄, about 0.03 to about 0.2 μM sodium selenite, about 0.4 to about 1.5 μM ZnSO₄, about 10000 to about 40000 μM D-glucose, about 3 to about 25 μM L-carnitine, about 6 to about 40 μM ethanolamine, about 30 to about 200 μM D(+)-galactose, about 80 to about 400 μM putrescine, and about 100 to about 400 μM sodium pyruvate, and essential fatty acids, hormones, and anti-oxidants in amounts effective for neuron growth. Preferably, the preferred fatty acids, hormones, and anti-oxidants comprise from about 0.001 to about 0.1 μM cortisol, from about 0.5 to about 16 μM reduced glutathione, from about 0.05 to about 20 μM linoleic acid, from about 0.1 to about 10 μM linolenic acid, from about 0.001 to about 0.1 μM progesterone, from about 0.02 to about 1 μM retinyl acetate, from 0 to about 0.6 μM 3,3′,5-triiodo-L-thyronine (T3), from about 0.1 to about 10 μM DL-α tocopherol, from about 0.1 to about 10 μM DL-α tocopherol acetate, from about 5 to about 200 μM human albumin, from about 0.001 to about 0.1 μM catalase, from about 0.1 to about 5 μM insulin, from about 0.01 to about 0.5 μM superoxide dismutase, and from about 0.01 to about 0.32 μM transferrin.

The sterile liquid aqueous medium contains a hydrogen ion buffer having a pKₐ of from about 6.9 to about 7.7 in an amount sufficient to maintain the pH in the desired range of about 6.9 to about 7.7 when in contact with neural tissue. Generally, the amount of the hydrogen ion buffer is about 5000 to about 25000 μM. Suitable buffers for use in the present invention include, 3-[N-morpholino]propane-sulfonic acid (MOPS), sodium bicarbonate, N-2-acetamido-2-aminoethanesulphonic acid (ACES), N,N-bis-(2-hydroxyethyl)-2-aminoethanesulphonic acid (BES), 1,3-diaza-2,4-cyclopentadiene, 2-tris(hydroxymethyl)aminoethanesulfonic acid (TES), and the like, as well as mixtures thereof. The preferred buffer in the base composition is 3-[N-morpholino]propane-sulfonic acid (MOPS). Sodium bicarbonate can play a dual role in the present invention. At low levels (i.e., generally less than about 2000 μM), sodium bicarbonate is involved in chloride ion transport. In addition, as noted, sodium bicarbonate can be employed as the buffer; when used as the buffer, sodium bicarbonate must, of course, be included at much higher levels (up to about 30,000 μM).

The medium of the present invention also contains effective amounts of at least ten essential amino acids. Preferably, the sterile liquid medium contains, in final concentration: (1) from about 250 to about 2500 μM each of L-isoleucine, L-leucine, L-lysine, L-threonine, and L-valine; (2) from about 150 to about 1500 μM L-glutamine; (3) from about 120 to about 1200 μM each of L-arginine, glycine, L-phenylalanine, L-serine, and L-tyrosine; (4) from about 60 to about 600 μM each of L-histidine and L-methionine; (5) from about 25 to about 250 μM each of L-tryptophan and L-proline; (6) from about 6 to about 60 μM L-alanine; (7) from about 3 to about 30 μM L-cysteine; (8) from about 1.5 to about 15 μM of L-asparagine; (9) from about 12 to about 120 μM i-inositol, (10) from about 10 to about 100 μM niacinamide; (11) from about 9 to about 90 μM choline chloride; (12) from about 6 to about 60 μM pyridoxal; (13) from about 2 to about 40 μM each of thiamine, folic acid, and D-Ca pantothenate; (14) from about 0.3 to about 3 μM riboflavin; (15) from about 0.1-1.2 μM biotin; and (16) from about 0.05 to about 1 μM vitamin B12.

Preferably, the growth-promoting fatty acids, hormones, and anti-oxidants comprise from about 0.002 to about 0.03 μM cortisol, from about 1 to about 8 μM reduced glutathione, from about 1 to about 10 μM linoleic acid, from about 0.2 to about 5 μM linolenic acid, from about 0.005 to about 0.06 μM progesterone, from about 0.05 to about 0.6 μM retinyl acetate, from about 0.0005 to about 0.2 μM 3,3′,5-triiodo-L-thyronine (T3), from about 0.5 to about 5 μM each of DL-α tocopherol and DL-α tocopherol acetate, from about 15 to about 90 μM human albumin, from about 0.002 to about 0.04 μM catalase, from about 0.2 to about 2 μM insulin, from about 0.02 to about 0.25 μM superoxide dismutase and from about 0.02 to about 0.16 μM transferrin.

The suggested components of the medium of this invention are indicated in the following Table 1:

TABLE 1

| | | Preferred Range | More Preferred |
|---|---|---|---|
| Components | Range (μM) | (μm) | Range (μM) |
| inorganic salts | | | |
| CaCl₂ | 0-3000 | 500-2500 | 1200-2400 |
| Fe(NO₃)₃.9H₂O | 0.1-1.2 | 0.05-0.6 | 0.1-0.3 |
| KCl | 2500-10000 | 3000-8000 | 4000-6000 |
| MgCl₂ | 0-4000 | 300-2000 | 600-1000 |
| NaCl | 30000-150000 | 40000-103000 | 66000-86000 |
| NaHCO₃ | 100-30000 | 200-1800 | 780-980 |
| NaH₂PO₄.H₂O | 250-4000 | 400-2000 | 800-1000 |
| sodium selenite | 0.01-0.4 | 0.03-0.2 | 0.06-0.1 |
| ZnSO₄.7H₂O | 0.2-2 | 0.4-1.5 | 0.57-0.77 |
| other | | | |
| D-glucose | 2500-50000 | 10000-40000 | 15000-35000 |
| MOPS | 5000-25000 | 8000-12000 | 9000-11000 |
| L-carnitine | 1-50 | 3-25 | 6-18 |
| ethanolamine | 3-80 | 6-40 | 12-20 |
| D(+)-galactose | 15-400 | 30-200 | 60-100 |
| human albumin | 5-200 | 15-90 | 30-45 |
| putrescine | 40-800 | 80-400 | 160-200 |
| sodium pyruvate | 20-500 | 100-400 | 130-330 |
| transferrin | 0.01-0.32 | 0.02-0.16 | 0.04-0.08 |
| amino acids | | | |
| L-alanine | 0-120 | 6-60 | 10-30 |
| L-arginine.HCl | 0-2400 | 120-1200 | 200-600 |
| L-asparagine.H₂O | 0-30 | 1.5-15 | 2.5-7.5 |
| L-cysteine | 0-60 | 3-30 | 5-15 |
| L-glutamine | 0-3000 | 150-1500 | 300-700 |
| glycine | 0-2400 | 120-1200 | 200-600 |
| L-histidine.HCl. | 0-1200 | 60-600 | 100-300 |
| H₂O | | | |
| L-isoleucine | 0-5000 | 250-2500 | 600-1000 |
| L-leucine | 0-5000 | 250-2500 | 600-1000 |
| L-lysine.HCl | 0-5000 | 250-2500 | 600-1000 |
| L-methionine | 0-1200 | 60-600 | 100-300 |
| L-phenylalanine | 0-2400 | 120-1200 | 200-600 |
| L-proline | 0-500 | 25-250 | 60-80 |
| L-serine | 0-2400 | 120-1200 | 200-600 |
| L-threonine | 0-5000 | 250-2500 | 600-1000 |
| L-tryptophan | 0-500 | 25-250 | 40-160 |
| L-tyrosine | 0-2400 | 120-1200 | 200-600 |
| L-valine | 0-5000 | 250-2500 | 600-1000 |
| antioxidants | | | |
| glutathione | 0.5-16 | 1-8 | 2-4 |
| (reduced) | | | |
| vitamin E | 0.1-10 | 0.5-5 | 1-3 |
| (α-tocoperol) | | | |
| vitamin E acetate | 0.1-10 | 0.5-5 | 1-3 |
| (α-tocoperol | | | |
| acetate) | | | |
| catalase | 0.001-0.1 | 0.002-0.04 | 0.005-0.02 |
| superoxide | 0.01-0.5 | 0.02-0.25 | 0.04-0.12 |
| dismutase | | | |
| hormones | | | |
| cortisol | 0.001-0.1 | 0.002-0.03 | 0.005-0.015 |
| DHEAS | 0-200 | 5-100 | 10-30 |
| progesterone | 0.001-0.1 | 0.005-0.06 | 0.01-0.03 |
| retinyl acetate | 0.02-1 | 0.05-0.6 | 0.1-0.3 |
| insulin | 0.1-5 | 0.2-2 | 0.4-0.8 |
| 3,3′,5-triiodo-L- | 0.0-0.6 | 0.0005-0.2 | 0.02-0.08 |
| thyronine (T3) | | | |
| essential fatty acids | | | |
| linoleic acid | 0.05-20 | 1-10 | 2.5-4.5 |
| linolenic acid | 0.1-10 | 0.2-5 | 0.5-2.0 |
| vitamins | | | |
| biotin | 0-2.5 | 0.1-1.2 | 0.2-0.6 |
| D-Ca pantothenate | 0-100 | 2-40 | 6-24 |
| choline chloride | 0-200 | 9-90 | 20-40 |
| folic acid | 0-100 | 2-40 | 4-14 |
| i-inositol | 0-240 | 12-120 | 20-60 |
| niacinamide | 0-200 | 10-100 | 15-50 |
| pyridoxal.HCl | 0-120 | 6-60 | 10-30 |
| riboflavin | 0-6 | 0.3-3 | 0.5-1.5 |
| thiamine.HCl | 0-100 | 2-40 | 5-20 |
| B12 (cobalamin) | 0-1.2 | 0.05-1 | 0.1-0.5 |

The sterile liquid medium of this invention may also contain an effective amount of basic fibroblast growth factor (basic FGF or FGF2) to assist in supporting survival and regeneration of neurons; generally an effective amount of FGF2 is about 1 to about 50 ng/ml and, more preferably, about 2 to about 20 ng/ml. An especially preferred FGF2 for use in the present invention is basic human recombinant fibroblast growth factor from Invitrogen, Inc. (Rockville, Md.).

It is generally preferred that all components are pharmaceutical grade or better. Moreover, for all human-derived components, it is generally preferred that, whenever possible, synthetic, or otherwise treated, components are used in order to reduce the risk of exposing patients to harmful agents (e.g., prions, HIV, and the like).

Hibernate™ with the addition of B27 supplement (U.S. Pat. No. 6,180,404) permits storage of viable brain tissue under refrigeration conditions for at least a month, thereby allowing shipment of viable brain tissue between laboratories. This sterile liquid medium with Neurobasal™ in place of Hibernate™ also supports regeneration in culture of adult hippocampal neurons of any age (Brewer, J. Neurosci. Meth., 1997; 71:143-155). From adult rat brains as old as 3 years (the human equivalent of 75 years), about 50 percent live cells can be isolated. In culture, these cells regenerate axons and dendrites, clearly demonstrating that adult neurons, after being axotomized during isolation, can regenerate in a sterile liquid medium. The improvements in this invention extend the use of such media during repair or injury to the damaged brain or spinal cord. Thus, this sterile liquid medium of the present invention is ideally suited for in vivo clinical preservation of neurons in the injured brain or other nervous system tissue.

The sterile liquid medium of this invention promotes the survival and regeneration of adult human neurons (Brewer et al., J. Neurosci. Methods, 2001; 107:15). Neuron characteristics of cytoskeletal immunoreactivity for neurofilament, MAP2 and tau, were demonstrated. Cultures could be maintained for many weeks in the sterile liquid medium of this invention, long enough to demonstrate the appearance of synaptic elements. Synaptic boutons, synaptic vesicles, and postsynaptic densities were observed. Due to the small number of cases, it has not been possible to identify factors other than the extent of marginal tissue source that may improve the frequency of regenerating neurons instead of glia. The combination of small tissue slices and sterile liquid medium of this invention may contribute to greater success. Earlier attempts with electrocauterized tissue did not produce viable neurons. The procedures presented herein, including partial hemostasis with pressure, and acquisition of tissue, followed by required electrocautery to the cut surface, should pose no burden on neurosurgery.

For the 40 percent of tissue samples in which neurons regenerated in culture, brain tissue experienced acute ischemia for several minutes before cooling in transport medium (Hibernate™/B27 as described in U.S. Pat. No. 6,180,404; www.siumed.edu/BrainBits). Viel et al. (J. Neurosci. Res., 2001; 64: 311) have shown that viable rat brain neurons can be obtained after total ischemia of up to 2 hours at room temperature. However, if the ischemic brain is cooled to 4° C., viable cortical neurons can be obtained after as long as 24 hours.

Synergism of DHEAS with FGF2 in improving survival of human cortical neurons when included in the sterile liquid medium of this invention has been demonstrated. FGF is a classic trophic factor for cortical neurons (Walicke et al., Proc. Natl. Acad. Sci., 1986; 83: 3012-3016; Morrison et al., Proc. Natl. Acad. Sci., 1986; 83: 7537-7541). Presynaptic neurons in the brain are thought to depend on these trophic factors released by appropriately innervated postsynaptic neurons. In extracting neurons from the thousands of synapses in the brain, it is not surprising that exogenous trophic support is needed to replace the FGF on which these neurons were depending. FGF binds to the FGF receptor on neurons (Walicke et al., J. Biol. Chem., 1989; 264: 4120-4126) to activate a tyrosine kinase cascade (Eckenstein, J. Neurobiol., 1994; 25:1467-1480). Part of a neuroprotective mechanism against glutamate toxicity in culture (Skaper et al., Dev. Brain Res., 1993; 71: 1-8; Mattson et al., J. Neurochem., 1995; 65: 1740-1751) as well as in vivo (Nakata et al., Brain Res., 1993; 605: 354-356) involves upregulation of anti-oxidant defenses (Mattson et al., J. Neurochem., 1995; 65:1740-1751). Therefore, there is ample precedent for the neuroprotective action of FGF. Brain-derived neurotrophic factor (BDNF) may provide additional trophic support (Kirschenbaum et al., Proc. Natl. Acad. Sci. U.S.A, 1995; 92: 210-214; Goldman et al., J. Neurobiol., 1997; 32: 554-566).

The mechanism of action of the steroid DHEAS in the sterile liquid medium of this invention is less clear. DHEAS enhances survival of isolated mouse cortical neurons and enhances learning after intracisternal injection (Roberts et al., Brain Res., 1987; 406: 357-362). Part of the mechanism involves protection from glutamate toxicity (Kimonides et al., Proc. Natl. Acad. Sci. U.S.A, 1998; 95: 1852-1857) by elevation of the neuroprotective transcription factor NF-kB (Mao et al., Neuro. Report, 1998; 9: 759-763), while inhibiting nuclear translocation of the glucocorticoid receptor (Cardounel et al., Proc. Soc. Exp. Biol. Med., 1999; 222: 145-149). DHEAS inhibits GABA-mediated chloride uptake in rat brain (Imamura et al., Biochem. Biophys. Res. Comm., 1998; 243:771-775) as well as rapidly blocking voltage-gated calcium currents in isolated hippocampal neurons (Ffrench-Mullen et al., Eur. J. Pharmacol., 1991; 202: 269-272). All of these activities could contribute to the beneficial effects of DHEAS on human and rat neuron survival reported here, but they suggest synergistic action of DHEAS with FGF2 by activation of a separate pathway to promote neuroprotection.

To the inventor's knowledge, high yields of adult human neurons have not been previously reported. Embryonic human neurons were cultured at least ten years ago (Mattson et al., Brain Res., 1990; 522: 204-214), but use of this tissue is not common due to scarcity of tissue and the ethical problem of a lack of informed consent of the tissue donor. Early reports of culture of adult human brain tissue as explants in serum-containing medium produced mainly astroglia (Gilden et al., Comp. Neurol., 1975; 161: 295-306), as judged by staining with GFAP (Gilden et al., J. Neurol. Sci., 1976; 29: 177-184). Silani et al. (Appl. Neurophysiol., 1988; 51: 10-20) also reported serum-containing explant cultures from two adults, but provided no immunocytological evidence for neurons. In another serum-containing culture, the yield of MAP2 positive neurons was only about 0.025 percent of plated cells (Kirschenbaum et al., Cereb. Cortex, 1994; 4: 576-589). Cells plated in the sterile liquid medium of this invention which contains both FGF2 and DHEAS yield about 20 percent neurons as indicated by immunostaining for neurofilament. This yield of 20 percent of plated cells is about 1000 times larger as compared to the sterile liquid medium of this invention without FGF2 and DHEAS. Brain cortex cells plated in the inventive medium yield 9,000 viable cells/mg, similar to the 10,000 cells/mg isolated from rat frontal cortex (Viel et al., J. Neurosci. Res., 2001; 64:311-321).

Tissue obtained from epilepsy cases and treated with the inventive composition may help to answer some fundamental questions about the disease: Will networks that develop in culture exhibit spontaneous bursting activity of a clonic or tonic nature? Will higher ratios of excitatory to inhibitory synapses redevelop or will the ratio of glutamatergic to gabaergic cells isolated vary with proximity to the epileptic focus? Alternatively, the regeneration of axons and dendrites in a new environment may lead to more normal network activity. Adult neurons are likely to have different characteristics than embryonic neurons (Brewer, Neurobiol. Aging, 1998; 19: 561-568; Evans et al., J. Neurosci. Meth., 1998; 79: 37-46; Collings et al., Brain Res. Bull., 1999; 48: 73-78). These adult human neurons may provide for superior human neuropharmacology, toxicology, and development of improved methods for brain grafts.

As noted above, the present invention provides a method to improve neural cell viability in brain or spinal cord tissue in humans after brain or spinal cord injury or surgery. The present method comprises applying or administering the sterile liquid medium, as described above, to the brain or spinal cord tissue involved in, or adjacent to, the brain or spinal cord tissue in need of treatment. Normally such brain or spinal cord tissue in need of treatment will be tissue associated with brain or spinal cord injury and/or surgery. In appropriate cases, the sterile liquid medium can be applied or administered to brain or spinal cord tissue in a prophylactic manner (e.g., priorto brain surgery). For purposes of this invention, “brain or spinal cord tissue” is intended, of course, to include tissue associated with the brain and spinal cord, but is also intended to include nerve associated tissue throughout the body. Thus, the sterile liquid medium of this invention can be used to increase nerve regeneration and/or nerve survival during, for example, surgical reattachment of severed limbs or other body parts or reconstruction of damaged limbs or other body parts involving nerve injury.

Various delivery systems are known and can be used to apply or administer the sterile liquid medium of this invention. The sterile liquid medium may be applied or administered by any convenient route, including, for example, infusion or bolus injection and may be administered together with other biologically active agents (e.g., antibiotics, growth factors, cytokines, anti-inflammatory agents, neurotransmitters, receptor agonists, antagonists, and the like). Generally, the preferred method of administration depends on the tissue to be treated and the particular situation with the patient. In specific embodiments, it may be desirable to administer the sterile liquid compositions of the invention locally to the area in need of treatment. This may be achieved by, for example, and not by way of limitation, local infusion during surgery, topical application (e.g., wound dressing), injection, catheter, or implant (e.g., implants, surgical packing, or filling materials formed from porous, non-porous, or gelatinous materials, including membranes, such as silicone-based membranes or fibers), and the like. In one embodiment, administration can be by direct injection at the site (or former site) of tissue to be treated.

The sterile liquid medium of this invention is especially adapted for use in surgical treatment of the brain, whether due to, for example, brain tumors, aneurisms, growths, stroke, or brain injury. The sterile liquid medium would normally be used to rinse or instill the surgical site and/or used to impregnate or saturate a surgical packing or filling material (e.g., implants formed from porous, non-porous, or gelatinous materials, including membranes, such as silicone-based membranes or fibers, and the like) intended to remain in the cavity after removal of the tumor or other nerve tissue. One preferred surgical packing material is a Gel-Foam™ sponge.

If desired, the sterile liquid medium can be delivered in a controlled release system over a period of time. Thus, for example, a pump connected to a reservoir containing the sterile liquid medium can be used. Alternatively, a polymeric or other filling material saturated with the sterile liquid medium by which the sterile liquid medium is released in a controlled manner can be used. Other controlled release systems can also be used. Such controlled release systems can, of course, be designed to allow the sterile liquid medium to be replenished as needed; moreover, using such a system, the composition of the sterile liquid medium can be modified over time to account for changes in the patient's condition and/or the rate of healing achieved.

As noted above, the present invention also provides a method for delivering of stem cells or nervous system cells or tissue having increased viability into a brain, spinal cord, or nervous system of a human, said method comprising (1) treating the stem cells or nervous system cells or tissue with an aqueous sterile liquid medium prior to or during the delivery of the stem cells or nervous system cells or tissue to the brain, spinal cord, or nervous system of the human and (2) delivering the treated stem cells or nervous system cells or tissue to the brain, spinal cord, or nervous system of the human, wherein the aqueous sterile liquid medium comprises 0 to about 3000 μM CaCl₂, about 0.01 to about 1.2 μM Fe(NO₃)₃, about 2500 to about 10000 μM KCl, 0 to about 4000 μM MgCl₂, about 30000 to about 150000 μM NaCl, about 100 to about 30000 μM NaHCO₃, about 250 to about 4000 μM NaH₂PO₄, about 0.01 to about 0.4 μM sodium selenite, about 0.2 to about 2 μM ZnSO₄, about 2500 to about 50000 μM D-glucose, about 1 to about 50 μM L-carnitine, about 3 to about 80 μM ethanolamine, about 15 to about 400 μM D(+)-galactose, about 40 to about 800 μM putrescine, about 20 to about 500 μM sodium pyruvate, and growth-promoting essential fatty acids, hormones, and anti-oxidants in amounts effective for neuron growth and wherein the aqueous sterile liquid medium has an osmolarity of from about 200 to about 270 mOsm, contains about 5000 to about 25000 μM of a hydrogen ion buffer having a pKₐ of from about 6.9 to about 7.7, and is essentially free of ferrous sulfate, glutamate, and aspartate. Cells, including stem cells, nervous system cells, or nervous system tissue treated with the aqueous sterile liquid medium of the present invention prior to or during the delivery of the cells to the brain, spinal cord, or nervous system of a human generally have increased viability and are more likelyto survive and/or reproduce in the brain, spinal cord, or nervous system. Even more preferably, the aqueous sterile liquid medium of the present invention can be used as a delivery system or carrier for implantation of such cells.

The following examples describe and illustrate the methods and compositions of the invention. These examples are intended to be merely illustrative of the present invention, and not limiting thereof in either scope or spirit. Unless indicated otherwise, all percentages are by weight. Those skilled in the art will readily understand that variations of the materials, conditions, and processes described in these examples can be used. All references referred to herein are hereby incorporated by reference.

GENERAL METHODS. Brain tissue was ethically obtained by informed consent from patients before surgery under protocols approved by the Springfield Committee for Research on Human Subjects and the UC Irvine IRB. No extra tissue was removed and samples did not conflict with the need for pathology specimens. Rather than using aspiration to remove all tissue, slices of tissue 1-3 mm thick were obtained from cortical access ports or tissue that was marginal to the lesion. Use of electrocautery was kept to a minimum to reduce oxyradicals and other neuron damage. Tissue obtained in the operating room (198±80 mg, mean±S.D., n=8) was transferred into 20 ml ice cold sterile transport medium in a sterile 50 ml polystyrene tube (Corning). The transport medium was Hibernate A (Brewer et al., NeuroReport, 1996; 7: 1509-1512) with 2 percent B27 medium supplement (Invitrogen, Inc., Rockville, Md.) (Brewer et al., J. Neurosci. Res., 1993; 35: 567-576) and 0.5 mM glutamine. Hibernate A contains D-glucose, pyruvate, balanced salts, amino acids, and vitamins. B27 contains 5 antioxidants and 15 other components. Although the transport medium preserves 50 percent of the viability of embryonic rat brain tissue for 4 weeks at 8° C., most of the results presented here used tissue stored no longer than 4 hours at 4° C. After transport on ice to a laminar flow sterile hood in the culture laboratory, meninges and white matter were removed from the tissue with a scalpel and forceps in a 35 mm dish in 2 ml transport medium. Tissue was cut into 0.5 mm slices, digested with papain, and dissociated into a single cell suspension (see Brewer, J. Neurosci. Methods, 1997; 71: 143-155). The cell suspension was enriched for neurons by centrifugation on a density gradient of Optiprep (Invitrogen). Optiprep was first diluted with 0.8 percent NaCl 50.5:49.5 (v/v, Optiprep:saline) to produce a density of 1.15 at 22° C. Later experiments showed superior performance of saline buffered with 10 mM MOPS, pH 7.4. The diluted Optiprep was further diluted with transport medium (v/v) to make a gradient containing 4 steps of 1 ml in a 15 ml centrifuge tube: bottom (0.35:0.65), 0.25:0.75, 0.2:0.8, top (0.15:0.85). Up to 6 ml of cell suspension was layered over the Optiprep step gradient. Although neurons and glia were present throughout the gradient, the fraction between the pellet and the dense band of debris was collected for the highest enrichment of neurons (Brewer, J. Neurosci. Methods, 1997; 71: 143-155). Cells were plated at a density of 320/mm² onto glass coverslips coated with polylysine as previously described. Cells were cultured in NeurobasalT™ A medium (Invitrogen) with 2 percent B27, 5 ng/ml FGF2 (basic human recombinant fibroblast growth factor, Invitrogen) and 0.5 mM glutamine in a humidified atmosphere of 9 percent O₂, and 5 percent CO₂ (Forma, Marietta, Ohio). In most experiments, DHEAS (dehydroepiandrosterone 3-sulphate, Sigma) was also included at 10 μg/ml. DHEAS was diluted from a stock of 1 mg/ml in 10 percent bovine serum albumin that was filter sterilized. EGF (20 ng/ml; murine, Invitrogen) and NT3 (100 ng/ml; recombinant, Regeneron) were added as indicated from 500×stock solutions in 1 percent BSA/PBS. Half of the medium was replaced with fresh medium every 3-4 days.

For immunocytology, cultures were rinsed twice in warm PBS and fixed for 10 minutes in 4 percent paraformaldehyde in PBS. After rinsing twice with PBS, cells for GFAP staining were postfixed for 20 minutes in acetic acid/ethanol (1:9, v/v) before rinsing again with PBS. Non-specific sites were blocked, and cells were permeabilized for 5 minutes in 5 percent normal goat serum, and 0.5 percent Triton X-100. Cells were incubated with primary antibodies for 1 hour at 22° C. In 5 percent goat serum, and 0.05 percent Triton as follows: mouse anti-neurofilament 200 (1:40, Sigma) with rabbit anti-cow GFAP (1:2000, Dako), or mouse anti-MAP2 (1:250, Boeringer-Mannheim) with rabbit anti-tau (1:2000). After rinsing four times with PBS, cells were incubated 1 hour at 22° C. with a rhodamine-conjugate of goat anti-rabbit IgG (heavy+light chain, 1:500, Tago) together with a cy2-conjugate of goat anti-mouse IgG (heavy+light chain, 1:100, Jackson). After rinsing four times in PBS, coverslips were mounted with Aquamount and imaged through a Nikon 60×/1.4 objective using a Spot cooled CCD camera (Diagnostic Instruments).

For electron microscopy (Deitch et al., J. Neurosci., 1993; 13:4301-4315), cells on coverslips were fixed for 1 hour at 37° C. with 2.5 percent glutaraldehyde, diluted directly into the culture medium from a stock of 25 percent. Without rinsing, cells were further fixed and stained for 0.5 hour at 37° C. with 0.1 percent OSO₄. Cells were rinsed with 0.125 M Na-phosphate, pH 7.4, and fixed further for 0.5 hour at 37° C. with 1 percent OSO₄. After rinsing with 3.6 percent NaCl and water, cells were stained for 1 hour with 5 percent aqueous uranyl acetate. After rinsing in water, cells were dehydrated in a graded series of ethanol, followed by propylene oxide. Cells were inverted into rubber molds filled with Spur's resin (Polysciences). After polymerization at 60° C. for 3 days to produce translucent discs 3 mm thick, areas of interest were visualized with a 20× objective and circled with a marking pen. Circled areas were transferred to the back side of the disc of resin with a scribe. The glass coverslip was removed by repeated exposure to liquid nitrogen and boiling water. Marked areas were removed with a saw and glued to metal stubs for trimming and sectioning. Ninety nm gold sections were deposited on slot grids coated with a film of formvar. Sections were stained with uranyl acetate and lead citrate and viewed in a Hitachi H7 microscope at 70 kV.

### EXAMPLE 1

Human cortical brain tissue was obtained for culture from eleven consecutive surgical cases, including seven tumor cases, three cases of epilepsy, and one case of suprabulbar palsy. Patients ranged in age from 41 to 70 years. Table 2 summarizes the results from these eleven cases.

TABLE 2
Human cortical brain surgical specimens for culture in B27/Neurobasal A

| | | | Tissue | Cell | | % Neurofilament, |
|---|---|---|---|---|---|---|
| Surgical | Condition/ | Age, | Weight | Yield | | % GFAP |
| Sample | Diagnosis¹ | Sex | (mg)² | (million)³ | Additions⁴ | (mean ± S.D.)⁵ |
| 1 | glioma, r. | 46, M | n.d. | 0.7 | 0 | 53 ± 10, 38 ± 7 |
| | temporal | | | | DHEAS | 70 ± 12, 31 ± 9 |
| | | | | | FGF2 | 67 ± 7, 30 ± 7 |
| 2 | tumor, r. | 69, M | 150 | 1.5 | FGF2 | <10%, >80% |
| | temporal | | | | | |
| 3 | glioma, l. | 41, M | 205 | 5.7 | FGF2 | 84 ± 8 |
| | frontal | | | | DHEAS | 61 ± 10 |
| | | | | | FGF2 + DHEAS | 73 ± 11 |
| 4 | epilepsy, l. | 33, F | 266 | 3.6 | FGF2 | 16 ± 5 |
| | superior temp. | | | | FGF2 + NT3 | 15 ± 2 |
| | gyrus | | | | FGF2 + EGF | 17 ± 4 |
| 5 | meningioma, r. | 70, M | 150 | 1.7 | FGF2 | 17 ± 10 |
| | temporal | | | | DHEAS | 25 ± 10 |
| | | | | | FGF2 + DHEAS | 65 ± 48 |
| 6 | epilepsy, l. | 53, F | 73 | 0.12 | FGF2 + DHEAS | 84 ± 6, 16 ± 6 |
| | temporal | | | | + testosterone | 85 ± 4, 15 ± 4 |
| | lobectomy | | | | 20 nM | |
| | | | | | + estradiol 18 nM | 89 ± 5, 11 ± 5 |
| 7 | r. temporal | 54, M | 323 | 1.2 | DHEAS, FGF2 | all glia |
| | cortex | | | | | |
| 8 | epilepsy | 15, F | 253 | 0.9 | DHEAS, FGF2 | all glia |
| 9 | primary brain | 67, F | 167 | 0.7 | DHEAS, FGF2 | 26 ± 3 |
| | lymphoma | | | | | |
| 10 | meningioma | 45, F | 349 | 1.9 | DHEAS, FGF2 | all glia |
| | l. temporal | | | | | |
| | lobe | | | | | |
| 11 | psuedobulbar | 53, F | 61 | 0.8 | DHEAS, FGF2 | all glia |
| | palsy | | | | | |

¹Source of cortical tissue and/or diagnosis of patient condition. l., left; r., right side of brain.
²Tissue was weighed before chopping and trituration; n.d., not determined.
³Cell yield was determined by exclusion of trypan blue after gradient isolation.
⁴Additions to culture medium: see General Methods.
⁵After 6 days, cultures were fixed and immunostained as described in General Methods. Four to six fields of 0.304 mm² were scored for positive immunoreativity to neurofilament. When two entries are shown in one row, the second value represents immunoreactivity to GFAP.

Neuronal Characteristics of Isolated Cells. Cytoskeletal components were used as specific markers to distinguish neurons from glia. Microtubule associated proteins, MAP2 and tau, are generally associated with somatodendritic and axonal regions of neurons, respectively. Neurofilament 200 is a common intermediate filament protein restricted to neurons, and GFAP is restricted to astroglia. All preparations were immunostained for at least one of these markers. As isolated from the frontal lobe of a 67 year old primary lymphoma case and cultured for six days, these cells showed neuron-like morphology and immunostained for neuron cytoskeletal elements, MAP2 and tau. Fine, branching processes are seen in the phase image. MAP2 staining reveals broad dendritic growth cones. One axon-like uniform narrow-caliber fiber is seen with tau staining as well as nuclear localization, typical of tau. As shown in Table 2, a majority of neurons over glia were recovered from three of six cases of glioma and two of three cases of epilepsy. The percentage of the cultures with neuronal staining characteristics ranged from 15 to 89 percent. Cells with fibers averaged 20 percent of the viable plated cells. Viable isolated cells were 9,000 cells/mg tissue (±2,000 S.E., n=10 from Table 2).

To provide additional morphological evidence for the neuronal nature of cells with the appearance of neurons, one preparation from a 70 year old meningioma case was cultured for 3 weeks to permit development of synapses before fixation for electron microscopy. Electron micrographs revealed uniform fibers with microtubules, characteristic of axons, and examples of apparent synaptic boutons with an abundance of uniform diameter clear vesicles. Postsynaptic densities are also evident. Not all of the contacts between fibers were found to have characteristics of synapses. Based on selection of regions of fiber contact by phase contrast light microscopy, subsequent electron microscopy indicated that the frequency of synapse formation was about 10 percent of fiber contacts, not unlike that seen with embryonic rat hippocampal neurons (Brewer, unpublished results).

The age of the patient did not correlate with the percentage of neurofilament positive cells (R²=0.03; data not shown). The types of tissue available from patients and provided by the neurosurgeons varied from malignancies to epilepsies. Fifty percent of the malignancy cases (3 of 6) and one of three epilepsy cases produced greater than 50 percent neurofilament positive cells. The number of cases is not large enough to reach definitive conclusions about the best patient material for cell culture.

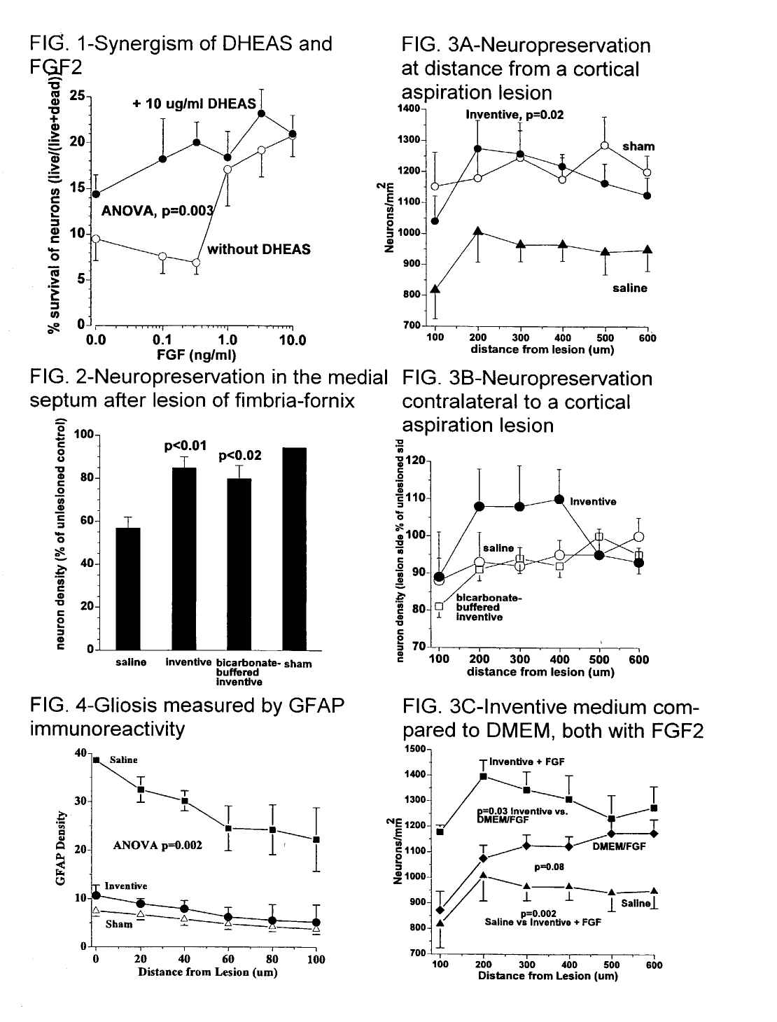

Requirements for Trophic and Hormonal Support. In preliminary experiments, the trophic requirement for FGF2 that was observed with adult rat neurons (Brewer, J. Neurosci. Methods, 1997; 71: 143-155) was evaluated for human neurons. For cells stained for neurofilament, FGF2 caused the percentage of positive cells to increase from 53±10 percent to 67±7 percent (t test, p<0.02, n=6 fields of about 20 cells/field, sample 1, Table 2). All subsequent experiments were performed in the presence of FGF2. In three cases, the ability of DHEAS at 10 μg/ml to promote survival was compared to FGF2. In each case, DHEAS was as effective as FGF2 in promoting survival of neurofilament positive, neuron-like cells. There was no apparent difference in morphology or neurofilament staining in adult human neurons cultured in the absence of FGF2 and DHEAS (A), or the presence of FGF2 (B), or DHEAS (C). In two cases, the combined effects of DHEAS and FGF2 on survival were no better than either one alone. However, at lower concentrations, DHEAS was synergistic for survival with FGF2 (FIG. 1). Without DHEAS, the ED₅₀ for survival was near 1 ng/ml FGF2. With DHEAS at 10 μg/ml, the ED₅₀ was lowered 10-fold to about 0.1 ng/ml FGF2 (FIG. 1). Concentrations of DHEAS at 1 and 3 μg/ml produced an intermediate survival between 0 and 10 μg/ml (data not shown). In one experiment (sample 4, Table 2), the trophic factors NT3 or EGF were added in addition to FGF2. These combinations were no more efficacious for survival of neurofilament positive cells than FGF2 alone. In another experiment (sample 6, Table 2), the sex hormones testosterone and estradiol were added to FGF2 and DHEAS in B27/Neurobasal without effect.

### EXAMPLE 2

During brain surgery, subarachnoid spaces, the brain parenchyma, and the resection cavity are generally rinsed with normal saline. Because normal saline is used to rinse the surgical field during human craniotomies and saline produces gliosis in rat cortical lesions (Gomez-Pinilla et al., J. Neurosci., 1995; 15: 2021-2029 ), the ability of normal saline to maintain neuron viability in a model system (i.e., rat embryonic hippocampal neurons in culture) was tested. After removal of the medium and treatment of these neurons with saline for 24 hours, more than half of the cells died, but all had lost dendritic processes. After 48 hours, saline caused nearly all cells to die. Parallel cultures treated with Neurobasal/B27 showed typical maximum survival of about 50 to 60 percent

To evaluate the effects of the sterile liquid medium of this invention in the brain, aspiration lesions of the rat cortex above the fimbria-fornix in rats were created with rinsing of the lesion with medium followed by implanting gelfoam soaked in the medium into the lesion cavity. Operations on 36 rats were carried out in a blinded protocol: each group of 6 rats received gelfoam soaked with one of the following compositions: (1) normal saline in the cortical lesion cavity; (2) the more preferred composition of Table 1 with the substitution of the rodent-appropriate corticosterone (0.01-0.05 μM) for cortisol; (3) Dulbecco's Modified Eagle's Medium (DMEM) with 10 ng/ml FGF2; (4) the more preferred composition of Table 1 with the substitution of the rodent-appropriate corticosterone (0.01-0.05 μM) for cortisol and with 5 ng/ml FGF2; and (5) control (i.e., no lesion or sham). Without knowledge of the treatment, a neuropathologist determined that lesion of the targeted fimbria-fornix was achieved in these rats. Neuron density 4 weeks after treatment was determined by counting cresyl violet stained neurons in the medial septum. A 55 percent improvement in neuron density was noted for treatments with the sterile liquid medium of this invention (FIG. 2). The improvement noted for treatment with the inventive medium was significantly greater than that seen with the saline treatment (p=0.01), and the density of neurons treated with the inventive medium approached the neuron density observed in the unlesioned sham cases. A similar formulation prepared with 26 mM sodium bicarbonate was slightly less effective (FIG. 2). Generally, the basic composition with the addition of FGF2 yielded results essentially the same as the basic composition alone in preserving neuron density in the medial septum. Thus, the basic composition of the sterile liquid medium described in the column labeled “more preferred range” in Table 1 above is generally preferred. These results emphasize the benefits of the composition of this invention to distant neurons whose axons have been severed or axotomized, as often occurs in brain or spinal cord surgery or head or spinal cord trauma.

### EXAMPLE 3

The preservation of neuron viability using the sterile liquid medium of this invention was tested in vivo using a brain lesion model. Aspiration (about 1 mm diameter) of rat cortex was performed to create a lesion cavity that was filled with a gelfoam sponge saturated with either (1) saline, (2) the preferred sterile liquid medium of this invention of composition listed in Table 1 (i.e., the first sterile liquid medium), or (3) the sterile liquid medium as in Table 1 buffered with 26 mM sodium bicarbonate buffer rather than MOPS buffer (i.e., the second sterile liquid medium). After 4 weeks of recovery, survival of cortical neurons was evaluated by fixation, embedding in paraffin, sectioning, and staining with cresyl violet. Survival of neurons surrounding the lesion was evaluated as a function of distance from the edge of the lesion and on the contralateral side. Images of the first 100 μm from the edge of the lesion suggests less cell loss with either sterile liquid medium of this invention used compared to saline. One month after surgery, rats treated with the first sterile liquid medium showed a mean 27 percent increase in preservation of neuron density above lesions treated with saline (split plot ANOVA with distance a within block variable, F(1, 10)=7.1, p=0.02) (FIG. 3A). Lesions treated with the bicarbonate buffered medium of this invention showed consistent benefits above saline, but did not reach significance (F(1, 10)=4.1, p=0.07). In comparison to sham lesioned animals (craniotomy without lesion), both the first and second sterile liquid mediums of this invention allow nearly full preservation of neuron density. However, the first sterile liquid medium generally provided better preservation.

As a percentage of neuron density in the same region of the unlesioned contralateral side of the cortex, lesions treated with the second (bicarbonate-buffered) sterile liquid medium were no different from those treated with saline (FIG. 3B). However, treatment with the first sterile liquid medium resulted in neuron densities even higherthan the unlesioned side over the middle distances of 200-400 μm (FIG. 3B). Over this distance, the first sterile liquid medium resulted in about a 15 percent improvement in neuron density over saline treatment, but did not reach significance (F(1, 4)=4, p=0.09). Contralateral loss of neurons induced by the lesion or changes in neuropil may be responsible for the lack of a larger difference. In sensorimotor cortical lesions, the contralateral neuropil has been shown to expand, which could cause a decrease in neuron density. Therefore, the raw data were reexamined for neuron density on the side contralateral to the lesion. A surprisingly large decrement of neuron density on the contralateral side was observed for saline treatment, while the first sterile liquid medium showed good preservation of neuron density on the contralateral side. These results further substantiate the benefits of the sterile liquid medium of this invention in preserving neuron density.

Rat lesions were also treated with the sterile liquid medium of Table 1 with the addition of FGF2 at 5 ng/ml to compare to a previously published treatment with DMEM with the addition of FGF2 at 10 ng/ml (Otto et al., J. Neurosci. Res., 1989; 22:83-91). FIG. 3C shows that the sterile liquid medium of the present invention with the addition of FGF2 caused a remarkable 50 percent increase in cell density above that seen with saline and significantly better than DMEM with the addition of FGF2 (p=0.03) as well as saline (p=0.002). These results indicate additional benefit of the combination of the sterile liquid medium with the addition of FGF2 as defined in this invention.

From the above treatments, sections were also deparifinized and immunostained for GFAP (glial fibrillary acidic protein) as an indication of the gliosis that occurs with brain injury. The density of immunostaining was measured as a function of distance from the edge of the aspiration lesion. FIG. 4 shows the high density of gliosis expected for lesions treated with saline (Gomez-Pinilla et al., J. Neurosci., 1995; 15: 2021-2029). Background levels of GFAP staining are indicated by the sham treatment. Significant reductions in lesion GFAP staining are seen in rat cortex treated with the medium of this invention (sterile liquid medium of this invention, p=0.002).

### EXAMPLE 4

The effects of the inventive medium on human tumor growth in culture was tested. Five consecutive tumors specimens were obtained from human patients undergoing craniotomy with lesion resection. Specimens were placed in sterile transport medium at 4° C. (Hibernate™/B27 as described in U.S. Pat. No. 6,180,404; www.siumed.edu/BrainBits) and shipped overnight on coldpacks. After 1 to 3 days of storage at 4° C., the tissue was chopped into 0.5 mm slices on a Mcllwain tissue chopper. Slices were digested for 30 minutes at 30° C. with papain (2 mg/ml, Worthington) in Hibernate A (BrainBits), followed by trituration in Hibernate A/B27/0.5 mM glutamine (GIBCO). The sample was divided into two portions and centrifuged for 1 minute at 200×g. One pellet was resuspended in the inventive medium with 0.5 mM glutamine; the other in Neurobasal A medium with 10% fetal bovine serum (GIBCO) and 0.5 mM glutamine. Viable cells were counted with trypan blue and plated in 2 cm² culture-treated polystyrene wells that had been precoated with 50 μg/ml poly-D-lysine in a 24 well plate. At one and at six or seven days after culture at 37° C. In 5% CO₂ and 9% O₂ (Thermo-Forma), phase-contrast images were acquired with a 20× Nikon objective through a Spot cooled CCD camera (Diagnostic Instruments). Isolated phase bright cells were counted in 12 adjacent fields of 0.373 mm², either by eye or with the assistance of Image-Pro+software (Media Cybernetics, Silver Spring, Md.). Mean cell densities per mm² of culture area are reported with standard errors. Between treatment t-tests were calculated with Plotit software (Scientific Programming Enterprises).

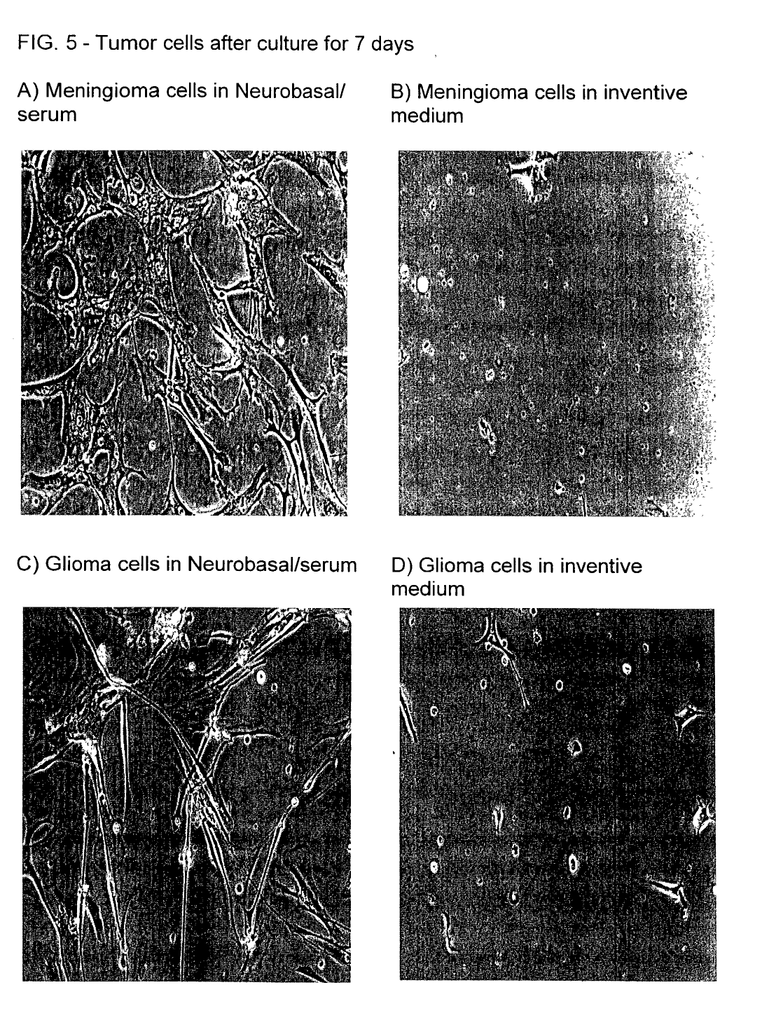

Meningioma cells grown in Neurobasal A medium with 10% fetal bovine serum (GIBCO) and 0.5 mM glutamine showed dramatic growth after 7 days. These cells spread onto the substrate and proliferated, reaching a mean cell area of 3015+453 μm² (mean+/−S.E., n=12 cells) (FIG. 5A). The same cells plated at the same density in the inventive medium after culture for 7 days did not spread or proliferate (mean area=936 μm²)(t-test, p=0.0001) (FIG. 5B);almost no live cells remained in the inventive medium.

Similarly, glioblastoma cells grown in Neurobasal A medium with 10% fetal bovine serum (GIBCO) and 0.5 mM glutamine spread onto the substrate and proliferated (FIG. 5C). By contrast, glioblastoma cells grown in inventive medium after culture for 7 days did not spread or proliferate (FIG. 5D).

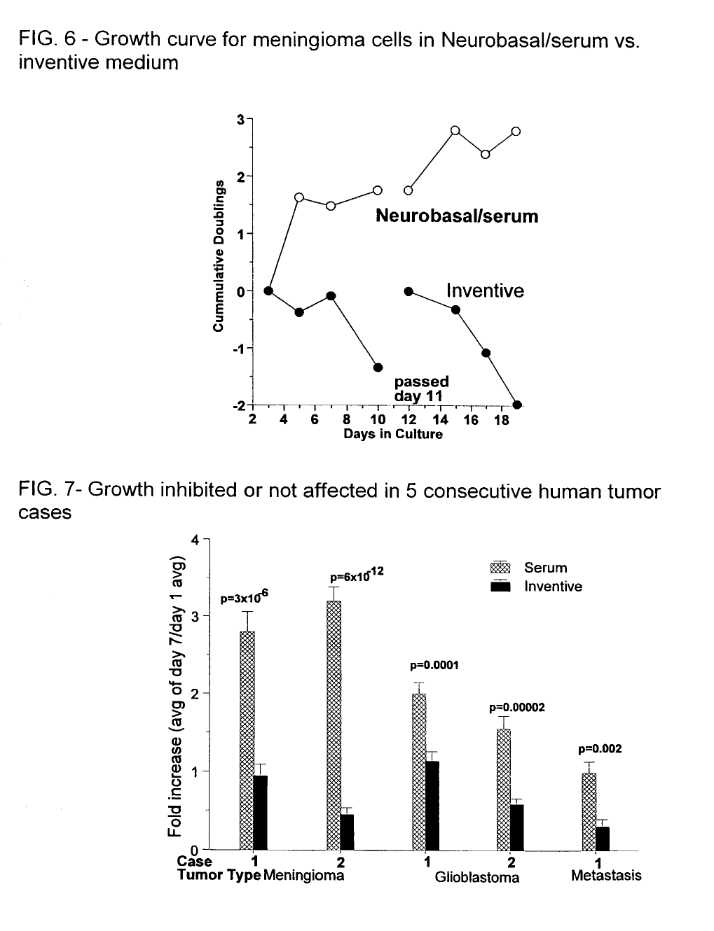

Cell growth for a meningioma case was followed over 10 days. After 10 days in culture, the cells grown in Neurobasal A medium with 10% fetal bovine serum (GIBCO) and 0.5 mM glutamine produced confluent growth. These cells were collected by trypsinization and replated either in Neurobasal A medium with 10% fetal bovine serum (GIBCO) and 0.5 mM glutamine or in inventive medium. Table 3, below, and FIG. 6 show that growth continued in Neurobasal A medium with 10% fetal bovine serum (GIBCO) and 0.5 mM glutamine serum, but growth was inhibited, and cells died in the inventive medium.

TABLE 3

+HC,12/ Cumulative population doublings

| | Neurobasal A/ | |
|---|---|---|
| days in culture | serum | Inventive Medium |
| 3 | 0 | 0 |
| 5 | 1.63 | −0.37 |
| 7 | 1.48 | −0.08 |
| 10 | 1.76 | −1.34 |

serum culture passed

| | | |
|---|---|---|
| 12 | 1.76 | 1.76 |
| 15 | 2.81 | 1.44 |
| 17 | 2.39 | 0.69 |
| 19 | 2.79 | −0.22 |

In Table 4, cell growth for various types of tumors are compared. The numbers in Table 4 represent the fold increase of cells at either six or seven days, calculated by dividing the number of cells at day 6 or 7 by the cell count at the start of the culture. These results are also shown in FIG. 7. For five consecutive tumor cases, the inventive medium results in growth stasis or inhibition and cell death, while Neurobasal A with fetal bovine serum caused cell proliferation in all primary tumors and cell stasis in the metastasis tumor.

TABLE 4

| | | Neurobasal | Inventive | |
|---|---|---|---|---|
| | | A/serum | Medium | |
| Tumor Type | Case | (S.E.) | (S.E.) | Probability |
| meningioma | 1 | 2.80 (0.26) | 0.94 (0.15) | 3 × 10⁻⁶ |
| | 2 | 3.19 (0.19) | 0.45 (0.09) | 6 × 10⁻¹² |
| glioblastoma | 1 | 2.00 (0.14) | 1.14 (0.12) | 1 × 10⁻⁴ |
| | 2 | 1.55 (0.17) | 0.59 (0.07) | 2 × 10⁻⁵ |
| metastasis | 1 | 0.99 (0.15) | 0.31 (0.09) | 2 × 10⁻³ |

As the experiments discussed above illustrate, the inventive medium inhibits human tumor cell growth in culture.

What is claimed is:

1. A method for improving neural cell viability in brain or spinal cord tissue in a human after brain or spinal cord injury or surgery, said method comprising applying an aqueous sterile liquid medium to the brain or spinal cord tissue, wherein the aqueous sterile liquid medium comprises 0 to about 3000 μM CaCl₂, about 0.1 to about 1.2 μM Fe(NO₃)₃, about 2500 to about 10000 μM KCl, 0 to about 4000 μM MgCl₂, about 30000 to about 150000 μM NaCl, about 100 to about 30000 μM NaHCO₃, about 250 to about 4000 μM NaH₂PO₄, about 0.01 to about 0.4 μM sodium selenite, about 0.2 to about 2 μM ZnSO₄, about 2500 to about 50000 μM D-glucose, about 1 to about 50 μM L-carnitine, about 3 to about 80 μM ethanolamine, about 15 to about 400 μM D(+)-galactose, about 40 to about 800 μM putrescine, about 20 to about 500 μM sodium pyruvate, and growth-promoting essential fatty acids, hormones, and anti-oxidants in amounts effective for neuron growth and wherein the sterile liquid medium has an osmolarity of from about 200 to about 270 mOsm, contains about 5000 to about 25000 μM of a hydrogen ion buffer having a pKₐ of from about 6.9 to about 7.7, and is essentially free of ferrous sulfate, glutamate, and aspartate.

2. The method as defined in claim 1, wherein the growth-promoting essential fatty acids, hormones, and anti-oxidants comprise about 0.001 to about 0.1 μM cortisol, about 0.5 to about 16 μM reduced glutathione, about 0.05 to about 20 μM linoleic acid, about 0.1 to about 10 μM linolenic acid, about 0.001 to about 0.1 μM progesterone, about 0.02 to about 1 μM retinyl acetate, 0 to about 0.6 μM 3,3′,5-triiodo-L-thyronine (T3), about 0.1 to about 10 μM DL-α tocopherol, about 0.1 to about 10 μM DL-α tocopherol acetate, about 5 to about 200 μM human albumin, about 0.001 to about 0.1 μM catalase, about 0.1 to about 5 μM insulin, about 0.01 to about 0.5 μM superoxide dismutase and, about 0.01 to about 0.32 μM transferrin.

3. The method as defined in claim 1, wherein the aqueous sterile liquid medium is buffered using 3-[N-morpholino]propane-sulfonic acid.

4. The method as defined in claim 2, wherein the aqueous sterile liquid medium is buffered using 3-[N-morpholino]propane-sulfonic acid.

5. The method as defined in claim 1, wherein the sterile aqueous medium further comprises (1) from about 0 to about 5000 μM each of L-isoleucine, L-leucine, L-lysine, L-threonine, and L-valine; (2) from about 0 to about 3000 μM L-glutamine; (3) from about 0 to about 2400 μM each of L-arginine, glycine, L-phenylalanine, L-serine, and L-tyrosine; (4) from about 0 to about 1200 μM each of L-histidine and L-methionine; (5) from about 0 to about 500 μM each of L-tryptophan and L-proline; (6) from about 0 to about 120 μM L-alanine; (7) from about 0 to about 60 μM L-cysteine; (8) from about 0 to about 30 μM of L-asparagine; (9) from about 0 to about 240 μM i-inositol, (10) from about 0 to about 200 μM niacinamide, (11) from about 0 to about 200 μM choline chloride, (12) from about 0 to about 120 μM pyridoxal, (13) from about 0 to about 100 μM each of thiamine, folic acid and D-Ca pantothenate, (14) from about 0 to about 6 μM riboflavin, (15) from about 0 to about 2.5 μM biotin, and (16) from about 0 to about 1.2 μM vitamin B12.

6. The method as defined in claim 2, wherein the aqueous sterile liquid medium further comprises (1) from about 0 to about 5000 μM each of L-isoleucine, L-leucine, L-lysine, L-threonine, and L-valine; (2) from about 0 to about 3000 μM L-glutamine; (3) from about 0 to about 2400 μM each of L-arginine, glycine, L-phenylalanine, L-serine, and L-tyrosine; (4) from about 0 to about 1200 μM each of L-histidine and L-methionine; (5) from about 0 to about 500 μM each of L-tryptophan and L-proline; (6) from about 0 to about 120 μM L-alanine; (7) from about 0 to about 60 μM L-cysteine; (8) from about 0 to about 30 μM of L-asparagine; (9) from about 0 to about 240 μM i-inositol, (10) from about 0 to about 200 μM niacinamide, (11) from about 0 to about 200 μM choline chloride, (12) from about 0 to about 120 μM pyridoxal, (13) from about 0 to about 100 μM each of thiamine, folic acid and D-Ca pantothenate, (14) from about 0 to about 6 μM riboflavin, (15) from about 0 to about 2.5 μM biotin, and (16) from about 0 to about 1.2 μM vitamin B12.

7. The method as defined in claim 1, wherein the aqueous sterile liquid medium further comprises dehydroepiandrosterone-4-sulfate.

8. The method as defined in claim 2, wherein the aqueous sterile liquid medium further comprises dehydroepiandrosterone-4-sulfate.

9. The method as defined in claim 7, wherein the dehydroepiandrosterone-4-sulfate is present in an amount of about 2 to about 200 μM.

10. The method as defined in claim 8, wherein the dehydroepiandrosterone-4-sulfate is present in an amount of about 2 to about 200 μM.

11. The method as defined in claim 1, wherein the aqueous sterile liquid medium further comprises human FGF2.

12. The method as defined in claim 2, wherein the aqueous sterile liquid medium further comprises human FGF2.

13. The method as defined in claim 8, wherein the aqueous sterile liquid medium further comprises human FGF2.

14. The method as defined in claim 10, wherein the aqueous sterile liquid medium further comprises human FGF2.

15. The method as defined in claim 12, wherein the human FGF2 is present in an amount of about 1 to about 20 ng/ml.

16. The method as defined in claim 13, wherein the human FGF2 is present in an amount of about 1 to about 20 ng/ml.

17. The method as defined in claim 15, wherein the human FGF2 is present in an amount of about 2 to about 10 ng/ml.

18. The method as defined in claim 16, wherein the human FGF2 is present in an amount of about 2 to about 10 ng/ml.

19. The method as defined in claim 1, wherein the aqueous sterile liquid medium is topically applied to the brain or spinal cord tissue by rinsing or instilling the brain or spinal cord tissue with the aqueous sterile liquid medium.

20. The method as defined in claim 2, wherein the aqueous sterile liquid medium is topically applied to the brain or spinal cord tissue by rinsing or instilling the brain or spinal cord tissue with the aqueous sterile liquid medium.

21. The method as defined in claim 1, wherein the aqueous sterile liquid medium is applied using a surgical packing material impregnated or saturated with the aqueous sterile liquid medium and wherein the impregnated or saturated surgical packing material is contacted with the brain or spinal cord tissue.

22. The method as defined in claim 2, wherein the aqueous sterile liquid medium is applied using a surgical packing material impregnated or saturated with the aqueous sterile liquid medium and wherein the impregnated or saturated surgical packing material is contacted with the brain or spinal cord tissue.

23. The method as defined in claim 1, wherein the aqueous sterile liquid medium is applied using a syringe or pump and wherein the aqueous sterile liquid medium is contacted with the brain or spinal cord tissue.

24. A method for delivering of stem cells or nervous system cells or tissue having increased viability into a brain, spinal cord, or nervous system of a human, said method comprising (1) treating the stem cells or nervous system cells or tissue with an aqueous sterile liquid medium prior to or during the delivery of the stem cells or nervous system cells or tissue to the brain, spinal cord, or nervous system of the human and (2) delivering the treated stem cells or nervous system cells or tissue to the brain, spinal cord, or nervous system of the human, wherein the aqueous sterile liquid medium comprises 0 to about 3000 μM CaCl₂, about 0.1 to about 1.2 μM Fe(NO₃)₃, about 2500 to about 10000 μM KCl, 0 to about 4000 μM MgCl₂, about 30000 to about 150000 μM NaCl, about 100 to about 30000 μM NaHCO₃, about 250 to about 4000 μM NaH₂PO₄, about 0.01 to about 0.4 μM sodium selenite, about 0.2 to about 2 μM ZnSO₄, about 2500 to about 50000 μM D-glucose, about 1 to about 50 μM L-carnitine, about 3 to about 80 μM ethanolamine, about 15 to about 400 μM D(+)-galactose, about 40 to about 800 μM putrescine, about 20 to about 500 μM sodium pyruvate, and growth-promoting essential fatty acids, hormones, and anti-oxidants in amounts effective for neuron growth and wherein the aqueous sterile liquid medium has an osmolarity of from about 200 to about 270 mOsm, contains about 5000 to about 25000 μM of a hydrogen ion buffer having a pKₐ of from about 6.9 to about 7.7, and is essentially free of ferrous sulfate, glutamate, and aspartate.

25. The method as defined in claim 24, wherein the growth-promoting essential fatty acids, hormones, and anti-oxidants comprise about 0.001 to about 0.1 μM cortisol, about 0.5 to about 16 μM reduced glutathione, about 0.05 to about 20 μM linoleic acid, about 0.1 to about 10 μM linolenic acid, about 0.001 to about 0.1 μM progesterone, about 0.02 to about 1 μM retinyl acetate, 0 to about 0.6 μM 3,3′,5-triiodo-L-thyronine (T3), about 0.1 to about 10 μM DL-α tocopherol, about 0.1 to about 10 μM DL-α tocopherol acetate, about 5 to about 200 μM human albumin, about 0.001 to about 0.1 μM catalase, about 0.1 to about 5 μM insulin, and about 0.01 to about 0.5 μM superoxide dismutase and, about 0.01 to about 0.32 μM transferrin; and wherein the aqueous sterile liquid medium further comprises about 2 to about 200 μM dehydroepiandrosterone 3-sulphate and about 1 to about 20 ng/ml human FGF2.

26. The method as defined in claim 24, wherein the treatment of the stem cells or nervous system cells or tissue with the aqueous sterile liquid medium is effected by storing or transferring the stem cells or nervous system cells or tissue in the aqueous sterile liquid medium.

27. The method as defined in claim 25, wherein the treatment of the stem cells or nervous system cells or tissue with the aqueous sterile liquid medium is effected by storing or transferring the stem cells or nervous system cells or tissue in the aqueous sterile liquid medium.

28. The method as defined in claim 24, wherein the treatment of the stem cells or nervous system cells or tissue with the aqueous sterile liquid medium is effected by combining the stem cells or nervous system cells or tissue and the aqueous sterile liquid medium prior to delivery and the aqueous sterile liquid medium is used as a vehicle for delivery of the treated stem cells or nervous system cells or tissue to the brain, spinal cord, or nervous system of the human.

29. The method as defined in claim 25, wherein the treatment of the stem cells or nervous system cells or tissue with the aqueous sterile liquid medium is effected by combining the stem cells or nervous system cells or tissue and the aqueous sterile liquid medium prior to delivery and the aqueous sterile liquid medium is used as a vehicle for delivery of the treated stem cells or nervous system cells or tissue to the brain, spinal cord, or nervous system of the human.

30. An aqueous composition effective for improving neural cell viability in brain or spinal cord tissue in a human after brain or spinal cord injury or surgery or for improving neural cell viability of nervous system cells or tissue intended to be delivered into a brain, spinal cord, or nervous system of a human, said aqueous composition comprising 0 to about 3000 μM CaCl₂; about 0.1 to about 1.2 μM Fe(NO₃)₃; about 2500 to about 10,000 μM KCl; 0 to about 4000 μM MgCl₂; about 30,000 to about 150,000 μM NaCl; about 100 to about 30,000 μM NaHCO₃; about 250 to about 4000 μM NaH₂PO₄; about 0.01 to about 0.4 μM sodium selenite; about 0.2 to about 2 μM ZnSO₄; about 2500 to about 50,000 μM D-glucose; about 1 to about 50 μM L-carnitine; about 3 to about 80 μM ethanolamine; about 15 to about 400 μM D(+)-galactose; about 5 to about 200 μM human albumin; about 40 to about 800 μM putrescine; about 20 to about 500 μM sodium pyruvate; about 0.01 to about 0.32 μM transferrin; 0 to about 120 μM L-alanine; 0 to about 2400 μM L-arginine; 0 to about 30 μM L-asparagine; 0 to about 60 μM L-cysteine; 0 to about 3000 μM L-glutamine; 0 to about 2400 μM glycine; 0 to about 1200 μM L-histidine; 0 to about 5000 μM L-isoleucine; 0 to about 5000 μM L-leucine; 0 to about 5000 μM L-lysine; 0 to about 1200 μM L-methionine; 0 to about 2400 μM L-phenylalanine; 0 to about 500 μM L-proline; 0 to about 2400 μM L-serine; 0 to about 5000 μM L-threonine; 0 to about 500 μM L-tryptophan; 0 to about 2400 μM L-tyrosine; 0 to about 5000 μM L-valine; about 0.5 to about 16 μM glutathione (reduced); about 0.1 to about 10 μM α-tocoperol; about 0.1 to about 10 μM α-tocoperol acetate; about 0.001 to about 0.1 μM catalase; about 0.01 to about 0.5 μM superoxide dismutase; about 0.001 to about 0.1 μM cortisol; 0 to about 200 μM DHEAS; about 0.001 to about 0.1 μM progesterone; about 0.02 to about 1 μM retinyl acetate; about 0.1 to about 5 μM insulin; 0 to about 0.6 μM 3,3′,5-triiodo-L-thyronine (T3); about 0.05 to about 20 μM linoleic acid; about 0.1 to about 10 μM linolenic acid; 0 to about 2.5 μM biotin; 0 to about 100 μM D-Ca pantothenate; 0 to about 200 μM choline chloride; 0 to about 100 μM folic acid; 0 to about 240 μM i-inositol; 0 to about 200 μM niacinamide; 0 to about 120 μM pyridoxal; 0 to about 6 μM riboflavin; 0 to about 100 μM thiamine; and 0 to about 1.2 μM cobalamin; and wherein the aqueous composition has an osmolarity of from about 200 to about 270 mOsm, contains about 5000 to about 25000 μM of a hydrogen ion buffer having a pKₐ of from about 6.9 to about 7.7, and is essentially free of ferrous sulfate, glutamate, and aspartate.

31. The aqueous composition as defined in claim 30, wherein the aqueous composition comprises about 500 to about 2500 μM CaCl₂; about 0.05 to about 0.6 μM Fe(NO₃)3; about 3000 to about 8000 μM KCl; about 300 to about 2000 μM MgCl₂; about 40,000 to about 103,000 μM NaCl; about 200 to about 1800 μM NaHCO₃; about 400 to about 2000 μM NaH₂PO₄; about 0.03 to about 0.2 μM sodium selenite; about 0.4 to about 1.5 μM ZnSO₄; about 10,000 to about 40,000 μM D-glucose; about 3 to about 25 μM L-carnitine; about 6 to about 40 μM ethanolamine; about 30 to about 200 μM D(+)-galactose; about 15 to about 90 μM human albumin; about 80 to about 400 μM putrescine; about 100 to about 400 μM sodium pyruvate; about 0.02 to about 0.16 μM transferrin; about 6 to about 60 μM L-alanine; about 120 to about 1200 μM L-arginine; about 1.5 to about 15 μM L-asparagine; about 3 to about 30 μM L-cysteine; about 150 to about 1500 μM L-glutamine; about 120 to about 1200 μM glycine; about 60 to about 600 μM L-histidine; about 250 to about 2500 μM L-isoleucine; about 250 to about 2500 μM L-leucine; about 250 to about 2500 μM L-lysine; about 60 to about 600 μM L-methionine; about 120 to about 1200 μM L-phenylalanine; about 25 to about 250 μM L-proline; about 120 to about 1200 μM L-serine; about 250 to about 2500 μM L-threonine; about 25 to about 250 μM L-tryptophan; about 120 to about 1200 μM L-tyrosine; about 250 to about 2500 μM L-valine; about 1 to about 8 μM glutathione (reduced); about 0.5 to about 5 μM α-tocoperol; about 0.5 to about 5 μM α-tocoperol acetate; about 0.002 to about 0.04 μM catalase; about 0.02 to about 0.25 μM superoxide dismutase; about 0.002 to about 0.3 μM cortisol; about 5 to about 100 μM DHEAS; about 0.005 to about 0.06 μM progesterone; about 0.05 to about 0.6 μM retinyl acetate; about 0.2 to about 2 μM insulin; about 0.0005 to about 0.2 μM 3,3′,5-triiodo-L-thyronine (T3); about 1 to about 10 μM linoleic acid; about 0.2 to about 5 μM linolenic acid; about 0.01 to about 1.2 μM biotin; about 2 to about 40 μM D-Ca pantothenate; about 9 to about 90 μM choline chloride; about 2 to about 40 μM folic acid; about 12 to about 120 μM i-inositol; about 10 to about 100 μM niacinamide; about 6 to about 60 μM pyridoxal; about 0.3 to about 3 μM riboflavin; about 2 to about 40 μM thiamine; about 0.05 to about 1 μM cobalamin; and about 1 to about 50 ng/ml human FGF2.

32. The aqueous composition as defined in claim 31, wherein the aqueous composition comprises about 1200 to about 2400 μM CaCl₂; about 0.1 to about 0.3 μM Fe(NO₃)₃; about 4000 to about 6000 μM KCl; about 600 to about 1000 μM MgCl₂; about 66,000 to about 86,000 μM NaCl; about 780 to about 980 μM NaHCO₃; about 800 to about 1000 μM NaH₂PO₄; about 0.06 to about 0.1 μM sodium selenite; about 0.57 to about 0.77 μM ZnSO₄; about 15,000 to about 35,000 μM D-glucose; about 6 to about 18 μM L-carnitine; about 12 to about 20 μM ethanolamine; about 60 to about 100 μM D(+)-galactose; about 30 to about 45 μM human albumin; about 160 to about 200 μM putrescine; about 130 to about 330 μM sodium pyruvate; about 0.04 to about 0.08 μM transferrin; about 10 to about 30 μM L-alanine; about 200 to about 600 μM L-arginine; about 2.5 to about 7.5 μM L-asparagine; about 5 to about 15 μM L-cysteine; about 300 to about 700 μM L-glutamine; about 200 to about 600 μM glycine; about 100 to about 300 μM L-histidine; about 600 to about 1000 μM L-isoleucine; about 600 to about 1000 μM L-leucine; about 600 to about 1000 μM L-lysine; about 100 to about 300 μM L-methionine; about 200 to about 600 μM L-phenylalanine; about 60 to about 80 μM L-proline; about 200 to about 600 μM L-serine; about 600 to about 1000 μM L-threonine; about 40 to about 160 μM L-tryptophan; about 200 to about 600 μM L-tyrosine; about 600 to about 1000 μM L-valine; about 2 to about 4 μM glutathione (reduced); about 1 to about 3 μM α-tocoperol; about 1 to about 3 μM α-tocoperol acetate; about 0.005 to about 0.02 μM catalase; about 0.04 to about 0.12 μM superoxide dismutase; about 0.005 to about 0.015 μM cortisol; about 10 to about 30 μM DHEAS; about 0.01 to about 0.03 μM progesterone; about 0.1 to about 0.3 μM retinyl acetate; about 0.4 to about 0.8 μM insulin; about 0.02 to about 0.08 μM 3,3′,5-triiodo-L-thyronine (T3); about 2.5 to about 4.5 μM linoleic acid; about 0.5 to about 2 μM linolenic acid; about 0.2 to about 0.6 μM biotin; about 6 to about 24 μM D-Ca pantothenate; about 20 to about 40 μM choline chloride; about 4 to about 14 μM folic acid; about 20 to about 60 μM i-inositol; about 15 to about 50 μM niacinamide; about 10 to about 30 μM pyridoxal; about 0.5 to about 1.5 μM riboflavin; about 5 to about 20 μM thiamine; about 0.1 to about 0.3 μM cobalamin; and about 2 to about 10 ng/ml human FGF2.

33. The aqueous composition as defined in claim 30, wherein the hydrogen ion buffer is 3-[N-morpholino]propane-sulfonic acid.

34. The aqueous composition as defined in claim 31, wherein the hydrogen ion buffer is 3-[N-morpholino]propane-sulfonic acid.

35. The aqueous composition as defined in claim 32, wherein the hydrogen ion buffer is 3-[N-morpholino]propane-sulfonic acid.
